(19) United States
(12) Patent Application Publication
(10) Pub. No.: US 2024/0285585 A1
(43) Pub. Date: Aug. 29, 2024

(54) L-ERGOTHIONEINE AND USES THEREOF

(71) Applicant: NANJING NUTRABUILDING BIO-TECH CO., LTD., Nanjing (CN)

(72) Inventors: Mingru WANG, Nanjing (CN); Ronghua YI, Nanjing (CN); Shawn WELLS, Nanjing (CN); Kylin LIAO, Nanjing (CN)

(21) Appl. No.: 18/422,930

(22) Filed: Jan. 25, 2024

Related U.S. Application Data
(63) Continuation of application No. PCT/CN2021/108809, filed on Jul. 28, 2021.

(51) Int. Cl. A61K 31/4172; A61P 37/04

(52) U.S. Cl. CPC A61K 31/4172; A61P 37/04

(57) ABSTRACT

The disclosure provides L-ergothioneine, novel forms of L-ergothioneine, and uses thereof, particularly for immune support and nutrient supplementation. In particular, the disclosure provides a method of improving a mammal's immune response to a microbial infection; an orally administered unit dosage form; and a method of inducing the production of reactive oxygen species in polymorphonuclear leukocytes in a mammal.

### CROSS REFERENCE TO RELATED APPLICATION

This application is a continuation application of International Patent Application No. PCT/CN2021/108809, filed on Jul. 28, 2021, the contents of which are incorporated herein by reference in its entirety.

### FIELD OF THE DISCLOSURE

The present disclosure relates to L-ergothioneine and to uses thereof.

### BACKGROUND OF THE DISCLOSURE

L-ergothioneine is a thiolhistidine derivative, first isolated from rye ergot (Claviceps purpurea) in 1909. It occurs naturally in common foods, including mushrooms, offal, cereals, and some varieties of black and red beans (Phaseolus vulgaris). It is chemically defined as (2S)-3-(2-thioxo-2,3-dihydro-1H-imidazol-4-yl)-2-(trimethylammonio)-propanoate, and has the following chemical structure:

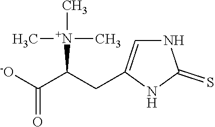

“Safety of synthetic 1-ergothioneine (Ergoneine®) as a novel food pursuant to Regulation (EC) No 258/97”, EFSA Journal (2016) 14(11).

Various sources of L-ergothioneine are marketed commercially, including a mushroom extract marketed by Blue California (Tomas, RSM, CA) and a chemically synthesized compound manufactured by Tetrahedron, Vincennes, France (U.S. Pat. No. 8,399,500 B2). A production process using a genetically modified S. Cerevisiae was recently proposed by van der Hoek S A et al. (2019) “Engineering the yeast Saccharomyces cerevisiae for the production of L-( [symbol] )-ergothioneine.” Front Bioeng Biotechnol 7, 262.

There is an urgent need for new antimicrobial agents. The use of reactive oxygen species (“ROS”) and oxygen free radicals as agents for antimicrobial mechanisms has been proposed based on their activity against Gram-positive and Gram-negative bacteria, viruses and fungi. ROS also can prevent and decompose biofilms. These functions make ROS very suitable for chronic inflammation, where antibiotics are often overused and relatively ineffective, in addition to chronic wounds, ulcers and burns; pulmonary disorders such as chronic sinusitis, chronic bronchitis, bronchiectasis, and cystic fibrosis; recurrent cystitis; and prosthetic device infection. Many clinical studies on ROS treatment are still to be carried out, but in vitro work on infection models and early clinical evaluation is very promising.

Polymorphonuclear leukocytes (“PMN”) are a type of white blood cell (“WBC”) that include neutrophils, eosinophils, basophils, and mast cells. Leukocytes (WBCs) are involved in protecting the body against infectious organisms, and PMNs are a subtype of leukocytes. Also known as granulocytes, PMNs play a central role in the innate immune system. PMNs are part of the non-specific innate immune system. This means that they treat all intruders in a similar fashion. The term innate means that this system can function from birth. The cells do not need to learn to recognize the invaders; they simply attack anything that the body considers foreign. A healthy PMN response can fight infection.

### SUMMARY OF DISCLOSURE

While studies to date have shown that L-ergothioneine is an antioxidant, the inventors have discovered just the opposite when the compound in the presence of PMNs. In particular, the inventors have discovered that at higher concentrations L-ergothioneine promotes the formation of ROS in PMNs, thereby providing novel approaches for supporting immune responses at relatively high doses of ergothioneine. These effects have been observed from a novel form of L-ergothioneine produced from S. Cerevisiae, not affected by the D-isomer if ergothioneine or the amino acid impurities (particularly any thiohistidine derivatives other than L-ergothioneine such as S-methyl-ergothioneine or selenium-containing selenoneine) that affect currently available sources of L-ergothioneine.

Thus, in a first principal embodiment, the disclosure provides a method of improving a mammal's immune response to a microbial infection comprising orally administering to the mammal an effective amount of L-ergothioneine or a pharmaceutically acceptable salt thereof, wherein: (a) the effective amount is sufficient to induce the production of reactive oxygen species in polymorphonuclear leukocytes; and (b) the L-ergothioneine comprises 0% D-ergothioneine, 0% nucleic acids, 0% amino acids, and less than 2% total impurities.

In a second principal embodiment, the disclosure provides a method of improving a mammal's immune response to a microbial infection comprising orally administering to the mammal an effective amount of L-ergothioneine or a pharmaceutically acceptable salt thereof, wherein the effective amount is sufficient to induce the production of reactive oxygen species in polymorphonuclear leukocytes.

In a third principal embodiment, the disclosure provides a method of supplementing L-ergothioneine stores in a mammal comprising orally administering to the mammal a supplement effective amount of L-ergothioneine or a pharmaceutically acceptable salt thereof, wherein the L-ergothioneine comprises 0% D-ergothioneine, 0% nucleic acids, 0% amino acids, and less than 2% total impurities.

In a fourth principal embodiment, the disclosure provides L-ergothioneine or a pharmaceutically acceptable salt thereof comprising 0% D-ergothioneine, 0% nucleic acids, 0% amino acids, and less than 2% total impurities.

In a fifth principal embodiment, the disclosure provides an orally administered unit dosage form comprising from 1 to 50 mg of L-ergothioneine or a pharmaceutically acceptable salt thereof comprising 0% D-ergothioneine, 0% nucleic acids, 0% amino acids, and less than 2% total impurities.

In a sixth principal embodiment, the disclosure provides a method of inducing the production of reactive oxygen species in polymorphonuclear leukocytes in a mammal comprising orally administering to the mammal an effective amount of L-ergothioneine or a pharmaceutically acceptable salt thereof.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE FIGURES

The accompanying drawings, which are incorporated in and constitute a part of this specification, illustrate several embodiments of the disclosure and together with the description serve to explain the principles of the disclosure.

FIG. 1 depicts the effects of L-ergothioneine on free radical (reactive oxygen species) formation by human polymorphonuclear phagocytes, when tested according to the methods of Example 1.

### DETAILED DESCRIPTION

### Definitions and Use of Terms

As used in the specification and claims, the singular forms a, an, and the include plural references unless the context clearly dictates otherwise. For example, the term “a specification” refers to one or more specifications for use in the presently disclosed methods and systems. “An ingredient” includes mixtures of two or more such ingredients, and the like. The word “or” or like terms as used herein means any one member of a particular list and also includes any combination of members of that list.

When used herein the term “about” will compensate for variability allowed for in the pharmaceutical industry and inherent in products in this industry, such as differences in product strength due to manufacturing variation and time-induced product degradation, as well as differences due to waters of hydration and different salts. The term also allows for any variation which in the practice of good manufacturing practices would allow the product being evaluated to be considered therapeutically equivalent or bioequivalent in humans to the recited strength of a claimed product. In some embodiments the term allows for any variation within 5% or 10% of the recited specification or standard.

As used in this specification and in the claims which follow, the word “comprise” and variations of the word, such as “comprising” and “comprises,” means “including but not limited to,” and is not intended to exclude, for example, other additives, components, integers or steps. When an element is described as comprising one or a plurality of components, steps or conditions, it will be understood that the element can also be described as “consisting of” or “consisting essentially of” the component, step or condition, or the plurality of components, steps or conditions.

“Effective amount” means that amount which, when administered to a human for supporting or affecting a metabolic process, or for treating or preventing a disease, is sufficient to cause such treatment or prevention of the disease or supporting or affecting the metabolic process. In any of the embodiments or subembodiments of this disclosure, an effective amount is capable of inducing the production of ROS, and preferably comprises 20 or 25 mg of L-ergothioneine or a pharmaceutically acceptable salt thereof when administered orally once daily.

When ranges are expressed herein by specifying alternative upper and lower limits of the range, it will be understood that the endpoints can be combined in any manner that is mathematically feasible. Thus, for example, a range of from 50 or 80 to 100 or 70 can alternatively be expressed as a series of ranges of from 50 to 100, from 50 to 70, and from 80 to 100. When a series of upper bounds and lower bounds are related using the phase “and” or “or”, it will be understood that the upper bounds can be unlimited by the lower bounds or combined with the lower bounds, and vice versa. Thus, for example, a range of greater than 40% and/or less than 80% includes ranges of greater than 40%, less than 80%, and greater than 40% but less than 80%. Unless otherwise specified by the term “between,” the boundaries of the range (lower and upper ends of the range) are included in the claimed range and can be preceded by the term “about.”

When an element of a process or thing is defined by reference to one or more examples, components, properties or characteristics, it will be understood that any one or any combination of those components, properties or characteristics can also be used to define the matter at issue. This might occur, for example, when specific examples of an element are recited in a claim (as in a Markush grouping), or an element is defined by a plurality of characteristics. Thus, for example, if a claimed system comprises element A defined by elements A1, A2 and A3, in combination with element B defined by elements B1, B2 and B3, the disclosure will also be understood to cover a system defined by element A without element B, a system in which element A is defined by elements A1 and A2 in combination with element B defined by elements B2 and B3, and all other possible permutations.

### Discussion of Principal Embodiments

In a first principal embodiment, the disclosure provides a method of improving a mammal's immune response to a microbial infection comprising orally administering to the mammal an effective amount of L-ergothioneine or a pharmaceutically acceptable salt thereof, wherein: (a) the effective amount is sufficient to induce the production of reactive oxygen species in polymorphonuclear leukocytes; and (b) the L-ergothioneine comprises 0% D-ergothioneine, 0% nucleic acids, 0% amino acids, and less than 2% total impurities.

In a second principal embodiment, the disclosure provides a method of improving a mammal's immune response to a microbial infection comprising orally administering to the mammal an effective amount of L-ergothioneine or a pharmaceutically acceptable salt thereof, wherein the effective amount is sufficient to induce the production of reactive oxygen species in polymorphonuclear leukocytes.

In a third principal embodiment, the disclosure provides a method of supplementing L-ergothioneine stores in a mammal comprising orally administering to the mammal a supplement effective amount of L-ergothioneine or a pharmaceutically acceptable salt thereof, wherein the L-ergothioneine comprises 0% D-ergothioneine, 0% nucleic acids, 0% amino acids, and less than 2% total impurities.

In a fourth principal embodiment, the disclosure provides L-ergothioneine or a pharmaceutically acceptable salt thereof comprising 0% D-ergothioneine, 0% nucleic acids, 0% amino acids, and less than 2% total impurities.

In a fifth principal embodiment, the disclosure provides an orally administered unit dosage form comprising from 1 to 50 mg of L-ergothioneine or a pharmaceutically acceptable salt thereof comprising 0% D-ergothioneine, 0% nucleic acids, 0% amino acids, and less than 2% total impurities.

In a sixth principal embodiment, the disclosure provides a method of inducing the production of reactive oxygen species in polymorphonuclear leukocytes in a mammal comprising orally administering to the mammal an effective amount of L-ergothioneine or a pharmaceutically acceptable salt thereof.

### Additional Embodiments

The disclosure can further be understood with reference to various subembodiments which can modify any of the principal embodiments. It will be understood that these subembodiments can be combined in any manner that is both mathematically and physically possible to create additional subembodiments, which in turn can modify any of the principal embodiments. Generally speaking, in any of the embodiments or subembodiments of the present disclosure, the patient scan be characterized as suffering from a general loss of appetite, early satiety, altered food preferences, or a combination thereof.

In some embodiments, particularly when L-ergothioneine is used to support immune function, the method is described based on the type of infection being treated. In preferred embodiments, the microbial infection is selected from the group consisting of bacterial infections, viral infections, fungal infections, and protozoal infections.

The L-ergothioneine also can be characterized by its purity or source of production. In any of the embodiments of the present disclosure, the L-ergothioneine preferably comprises 0% D-ergothioneine, 0% nucleic acids (particularly any thiohistidine derivatives other than L-ergothioneine such as S-methyl-ergothioneine or selenium-containing selenoneine), 0% amino acids, and less than 2% total impurities. The L-ergothioneine can also be characterized by other aspects of its purity, and in various embodiments comprises less than 0.5%, 0.1%, 0.05%, or 0.01%, of the disulfide of L-ergothioneine.

Depending on the purity profile intended, various methods of manufacturing are disclosed in the prior art that can be used to manufacture the L-ergothioneine used in the current disclosure, including a chemical synthesis process described by Tetrahedron, Vincennes, France in U.S. Pat. No. 8,399,500 B2, a genetically modified S. Cerevisiae process described by van der Hoek S A et al. (2019) “Engineering the yeast Saccharomyces cerevisiae for the production of L-( [symbol] )-ergothioneine.” Front Bioeng Biotechnol 7, 262, and an E. Coli process described by Nanjing Nutrabuilding Bio-tech Co., Ltd. in WO 2021/102736 A1.

The methods are generally practiced using doses of L-ergothioneine sufficient to induce the production of reactive oxygen species in polymorphonuclear leukocytes. The dose of L-ergothioneine can vary across a range of suitable doses depending on the health of the subject, the desired response, the dosage form and the route of administration. In a preferred subembodiment when administration is oral, the effective amount is from about 15 to about 50 mg/day of L-ergothioneine, from about 15 to about 35 mg/day, preferably from about 20 to about 30 mg/day, and most preferably about 20 or about 25 mg/day. In a preferred specific embodiment when administration is oral, the effective amount is from about 1 to about 500 mg/day of L-ergothioneine, from about 1 to about 300 mg/day, from about 1 to about 100 mg/day, from about 1 to about 50 mg/day, from about 3 to about 50 mg/day, from about 5 to about 50 mg/day, from about 10 to about 50 mg/day, from about 10 to about 30 mg/day. The dose is preferably administered as a single administration once per day, thus comprising 15-50 mg of L-ergothioneine, 15-35 mg of L-ergothioneine, 20-30 mg of L-ergothioneine, or 20 or 25 mg of L-ergothioneine. The L-ergothioneine is preferably administered as an oral dosage form comprising 1-500 mg, 1-300 mg, 1-100 mg, 1-50 mg, 3-50 mg, 5-50 mg, 10-50 mg, 10-30 mg of L-ergothioneine. The most preferred form of the compound is free acid, and the foregoing doses are preferably based on the weight of the free acid.

### Dosage Forms/Routes of Administration

Pharmaceutical compositions for preventing and/or treating a subject are further provided comprising an effective amount of L-ergothioneine or a pharmaceutically acceptable salt or adduct thereof and one or more pharmaceutically acceptable excipients. A “pharmaceutically acceptable” excipient is one that is not biologically or otherwise undesirable, i.e., the material can be administered to a subject without causing any undesirable biological effects or interacting in a deleterious manner with any of the other components of the pharmaceutical composition in which it is contained. The carrier can be selected to minimize any degradation of the active ingredient and to minimize any adverse side effects in the subject, as would be well known to one of skill in the art. The carrier can be a solid, a liquid, or both.

The disclosed compounds can be administered by any suitable route, preferably in the form of a unit dosage form adapted to such route, and in a dose effective for the treatment or prevention intended. For oral administration, the L-ergothioneine and other ingredients can be enclosed in a hard or soft-shell gelatin capsule, compressed into tablets, or incorporated directly into the individual's diet. Suitable dosage forms include ingestible tablets, buccal tablets, films, powder sachets, troches, capsules, elixirs, suspensions, syrups, wafers, and the like. Such compositions and preparations should contain at least 1% by weight of active compound. The percentage of the compositions and preparations can, of course, be varied and can conveniently be from about 5% to about 80% of the weight of the unit.

Tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor.

### EXAMPLES

In the following examples, efforts have been made to ensure accuracy with respect to numbers (e.g., amounts, temperature, etc.) but some errors and deviations should be accounted for. The following examples are put forth to provide those of ordinary skill in the art with a complete disclosure and description of how the methods claimed herein are made and evaluated and are intended to be purely exemplary of the disclosure and are not intended to limit the scope of what the inventors regard as their disclosure.

### Example 1. Testing of L-Ergothioneine

L-ergothioneine meeting the purity specifications defined herein was tested using in an in vitro PMN model using cells from a healthy blood donor. Minute samples of blood were exposed to L-ergothioneine (0.01-5 g/L) for 24 hours. The cells were then loaded with the dihydrorhodamine dye, which turns fluorescent upon exposure to ROS. Formation of ROS was triggered by addition of the bacterial peptide f-Met-Leu-Phe (fMLP). The samples were treated with a lysing buffer to eliminate most of the red blood cells in the samples, after which the samples were fixed in formalin. The fluorescence intensity of the PMN cells was evaluated by flow cytometry. The low fluorescence intensity of untreated control cells served as a baseline and PMN cells treated with fMLP alone served as a positive control. If the fluorescence intensity of PMN cells exposed to a test product was increased compared to fMLP alone, this indicated that a test product has pro-inflammatory effects by enhancing this aspect of anti-microbial immune defense mechanisms.

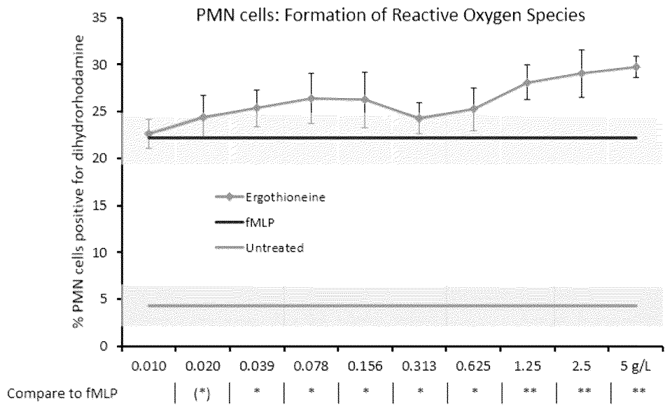

The results are depicted in FIG. 1. In FIG. 1, the solid grey line shows the average of untreated cells, and the lighter grey shade covering the grey line shows the standard deviation for untreated cells. The solid black line shows the average of cells activated by the bacterial peptide fMLP, and the lighter grey shade covering the black line shows the standard deviation for activated cells. The percent inhibition is shown as the average±standard deviation of sextuplicate data points for each dose of L-ergothioneine.

### Synopsis:

L-ergothioneine enhanced ROS formation in response to the bacterial peptide fMLP, suggesting that L-ergothioneine enhances this aspect of anti-microbial immune defense mechanisms.
At the doses tested there was an increase in ROS formation.
This increase reached a high level of significance for the highest 3 doses (p<0.01).
The increase remained statistically significant across the 0.6 g/L dose reported in human tissue when compared to the control cultures.

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of improving a mammal's immune response to a microbial infection comprising orally administering to the mammal an effective amount of L-ergothioneine or a pharmaceutically acceptable salt thereof, wherein the effective amount is sufficient to induce the production of reactive oxygen species in polymorphonuclear leukocytes.

2. The method of claim 1, wherein the L-ergothioneine comprises 0% D-ergothioneine, 0% nucleic acids, 0% amino acids, and less than 2% total impurities.

3. The method of claim 1, wherein the microbial infection is selected from the group consisting of bacterial infections, viral infections, fungal infections, and protozoal infections.

4. The method of claim 1, wherein the effective amount comprises from 1 to 100 mg/day of L-ergothioneine or a pharmaceutically acceptable salt thereof.

5. The method of claim 1, wherein the L-ergothioneine is administered as an oral dosage form comprising from 15 to 50 mg of L-ergothioneine.

6. The method of claim 1, wherein the L-ergothioneine is administered as an oral dosage form comprising about 20 mg or about 25 mg of L-ergothioneine.

7. The method of claim 1, wherein the mammal is a human.

8. An orally administered unit dosage form comprising from 1 to 50 mg of L-ergothioneine or a pharmaceutically acceptable salt thereof comprising 0% D-ergothioneine, 0% nucleic acids, 0% amino acids, and less than 2% total impurities.

9. The unit dosage form of claim 8, in the form of a tablet, capsule, powder sachet, or liquid.

10. The unit dosage form of claim 8, comprising about 20 mg or about 25 mg of L-ergothioneine.

11. The unit dosage form of claim 8, wherein the unit dosage form is used for improving a mammal's immune response to a microbial infection.

12. The unit dosage form of claim 11, wherein the microbial infection is selected from the group consisting of bacterial infections, viral infections, fungal infections, and protozoal infections.

13. The unit dosage form of claim 8, wherein the unit dosage form is used for inducing the production of reactive oxygen species in polymorphonuclear leukocytes in a mammal.

14. A method of inducing the production of reactive oxygen species in polymorphonuclear leukocytes in a mammal comprising orally administering to the mammal an effective amount of L-ergothioneine or a pharmaceutically acceptable salt thereof.

15. The method of claim 14, wherein the L-ergothioneine comprises 0% D-ergothioneine, 0% nucleic acids, 0% amino acids, and less than 2% total impurities.

16. The method of claim 14, wherein the method is used for improving the mammal's immune response to a microbial infection, the microbial infection is selected from the group consisting of bacterial infections, viral infections, fungal infections, and protozoal infections.

17. The method of claim 14, wherein the effective amount comprises from 1 to 100 mg/day of L-ergothioneine or a pharmaceutically acceptable salt thereof.

18. The method of claim 14, wherein the L-ergothioneine is administered as an oral dosage form comprising from 1 to 50 mg of L-ergothioneine.

19. The method of claim 14, wherein the L-ergothioneine is administered as an oral dosage form comprising about 20 mg or about 25 mg of L-ergothioneine.

20. The method of claim 14, wherein the mammal is a human.
